(19) United States
(12) Patent Application Publication
(10) Pub. No.: US 2004/0106093 A1
(43) Pub. Date: Jun. 3, 2004

(54) Myb-transformed blood cells and their use for active ingredient screening

(76) Inventors: Petr Bartunek, Prague (CZ); Michal Dvorak, Prague (CZ); Martin Zenke, Schoenow (DE); Vit Karafiat, Prague (CZ)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/471,343

(22) PCT Filed: Mar. 8, 2002
(86) PCT No.: PCT/DE02/00816

(30) Foreign Application Priority Data
Mar. 8, 2001 (DE) 101 12 360.4

(51) Int. Cl. C12N 5/08; A01N 1/00

(52) U.S. Cl. 435/1.1; 435/372; 435/375

(57) ABSTRACT

The invention relates to transformed blood cells and their use for active ingredient screening. The field of application of the invention are the pharmaceutical industry and medicine.

It is the object of the invention to develop hematopoietic cells that are suitable for an active ingredient screening.

It was found that v-Myb, a retrovirally transduced version of c-Myb, transforms primitive erythroid precursor cells (in the following designated as Myb/bFGF-cells) in the presence of the growth factor bFGF (basis fibroblast growth factor). The reason for this are both the features of v-Myb that are taken over from c-Myb, and new properties developed in interaction with bFGF.

The invention is based on transformed blood cells for elucidation of factors, components that influence the formation and differentiation of blood cells, which are characterised in that they contain Myb, in particular v-Myb, and growth factors that interact with Myb.

The invention relates to transformed blood cells and their use for active ingredient screening. The field of application of the invention are the pharmaceutical industry and medicine.

The development of hematopoietic cells is controlled by several factors. Signals of extracellular proteins in interplay with signals of specific intracellular regulators determine the phenotype of the individual hematopoietic cell. One of the regulators that holds a key role is c-Myb that is essential for the propagation of immature erythroid and myeloid cells. Furthermore, c-Myb stands in interaction with extracellular signals and thereby influences the further fate of the hematopoietic cells (Weston, K., 1998, Curr Opin Genet Dev, 8, 76-81).

It is the object of the invention to develop hematopoietic cells that are suitable for an active ingredient screening.

The invention is realised according to the claims 1, 8, and 9, the dependent claims are preferred variants.

It was found that v-Myb, a retrovirally transduced version of c-Myb, transforms primitive erythroid precursor cells (in the following designated as Myb/bFGF-cells) in the presence of the growth factor bFGF (basis fibroblast growth factor). The reason for this are both the features of v-Myb that are taken over from c-Myb, and new properties developed in interaction with bFGF.

Myb/bFGF-cells stem from precursor cells of the erythropoiesis and grow in a bFGF-dependent manner into a homologous cell population with a large number of cells. Proliferation rates of 10⁷ are easily reached. The resulting cells possess the initial differentiation potential and can grow into fully mature erythrocytes within 3 to 4 days. This differentiation nevertheless can be fully blocked by bFGF. This indicates that bFGF develops its activity in dependency of phospholipase C gamma by involving specific inhibitors.

Therefore, the described in vitro system represents a tool for the identification of new components that are both involved in the development of the proliferation pool of the red blood corpuscles and can play a role during their differentiation. This in vitro system in addition opens the possibility to identify components that are able to increase the proliferation of the red blood corpuscles and finally can be used for the treatment of leukaemias, blood loss by operations or injuries or in the fatigue syndrome.

On the other hand, the components that are identified with the aid of this system that accelerate the differentiation of the red blood corpuscles and/or induce the cell death can be used for the treatment of leukaemias.

The invention shall in the following illustrated in more detail by embodiments.

1. Methods

Cells and Cell Culture

Chicken embryo fibroblasts (CEF) were grown in Dulbecco's modified Eagle's-Medium (DMEM), supplemented with 8% foetal calf serum (FCS; Sebak, Switzerland) and 2% chicken serum (ChS; Sigma), 20 mM HEPES pH 7.3 and 100 units/ml Penicillin/Streptomycin (Gibco-BRL). Retroviral vector DNA (10 μg) pNeoAMV and pNeoCCC (Lipsick et al, 1986) containing v-myb and c-myb, respectively, were transfected into CEF (together with 1 μg MAV-1 helper virus DNA, pATMAV-1; Pecenka et al. 1988) and neo-resistant virus producing cells were selected (800 μg/ml G418; Gibco-BRL).

Blastoderm-derived cells (2 days incubation, Hamilton-stage HH 10-12) and bone marrow cells (4-7 old chicken) were cocultivated for 2 days with a mitomycin C-treated virus or control (non-infected)-CEF in a CFU-E-medium (Beug et al., 1995, Methods Enzymol, 254, 41-76) containing bFGF (25 ng/ml, Promega), chicken-SCF (100 ng/ml; Bartunek et al., 1996, Cytokine, 8, 14-20), TGFα (5 ng/ml, Promega) or different combinations thereof, or no factors. After 2 days nonadherent cells were regenerated and cells were grown at 2×10⁶ cells/ml. An outgrowth of myb transformed cells was routinely observed at day 5-7 and cell numbers were determined in regular time intervals using the cell counter and analyser system CASY-1 (Scharfe System, Reutlingen, Germany).

Proliferation Assays

Cell proliferation was measured as ratio of DNA-synthesis by ³H-thymidin-incorporation or use CellTiter96® (Promega). Briefly, the cells (4×10⁴ cells/100 μl) were incubated in CFU-E-Medium (see above) with 0.8 μCi ³H-thymidine (specific activity 29 Ci/mmol; Amersham, UK) for 2 hours at 37° C., brought onto filter plates and subjected to scintillation counting. The average values from triplicate samples were determined. The Celltiter96 MTS-assay was for 2 hours. Heparin (B. Braun, Melsungen, Germany) was used at 5 μg/ml. Inhibitors were used at the following concentrations: PD98059 (5 μM), PP2 (100 nM), U-73122 (1 μM), Worthmannin (100 nM), Cyclosporin A (10 nM), GF 109203X (10 nM) and KN-62 (1 μM) (all from Calbiochem).

Differentiation Assay

To produce erythroid differentiation, cells were incubated in CFU-E-medium without ChS (2×10⁶ cells/ml) supplemented with 3% anaemic chicken serum (as source for erythropoietin) plus 10 ng/ml recombinant human insulin (Novo Nordisk) in the presence or absence of 25 ng/ml bFGF. Erythroid differentiation was assessed by (1) determining haemoglobin accumulation in cytospin preparations that stained with neutral, and Diff-Quik (Baxter, Switzerland), or in haemoglobin assay (Beug et al., 1995, Methods Enzymol, 254, 41-76) (2), measuring reduction in cell size with the CASY-1 Cell Counter and Analyser System; and (3) loss of proliferative potential in ³H-thymidine incorporation assays. Photographs were taken and processed as above.

Flow Cytometry

Surface antigen expression was analysed by flow cytometry. 10⁶ cells were recovered, washed in PBS containing 1% bovine serum albumin (BSA, Fraktion V, Sigma) and incubated with specific monoclonal antibodies (1 h), followed by reaction with FITC-conjugated anti-mouse IgG (Fc specific; 45 min; Jackson Laboratories). The following antibodies were used:: MC51-2, MC47-83; JS4, JS8 (Schmidt et al., 1986, Exp. Cell Res, 164, 71-78). Cells were washed twice and resuspended in PBS containing 1% BSA and propidium iodide (2 μg/ml; Sigma). For flow cytometry analysis Calibur FACScan device with CellQuest-Software (Becton Dickinson) were used.

2. Results

bFGF Causes Growth of Primitive Erythroid Cells Containing c-Myb and v-Myb.

Primitive and definitive hematopoietic cells were produced from blastoderm of 2 day old chicken embryos or bone marrow, respectively, and infected with c-Myb and v-Myb expressing retroviral vectors. Cells were then cultured with bFGF plus SCF or with bFGF alone, and cumulative cell numbers were determined in regular time intervals. In cultures of primitive blastoderm derived cells, an outgrowth of immature blastoderm-like cells was observed at day 5-7 with bFGF plus SCF and bFGF alone, and cells grew for more than 50 days (FIG. 1A). In these cultures v-myb-cells showed higher proliferation rates than c-myb-cells. No outgrowth was observed in non-infected control cultures irrespective of the presence or absence of bFGF and SCF. Thus, it appears that the proliferation of primitive blastoderm derived cells was critically dependent on the presence of bFGF and Myb-expression. These cells will in the following be referred to as bFGF/c-myb or bFGF/v-myb progenitors.

In parallel experiments performed with infected bone marrow cells, an initial out-growth of blast-like cells was also seen in the absence of myb infection (FIG. 1B). This was not unexpected since under these conditions both myeloid and erythroid SCF dependent cells would transiently grow (Dolznig et al., 1995, Cell Growth Differ, 6 1341-1352). However, from day 10 onwards only c-myb and v-myb containing cultures proliferated.

At day 18 of culture cells were subjected to cytochemical analysis. Blastoderm derived bFGF/v-myb-cells exhibited an erythroid progenitor-like phenotype which was even more pronounced in bFGF/c-myb-cells where partially and terminally differentiated erythroid cells were observed. In bone marrow derived cultures v-myb-cells clearly represented mono-blast-like cells and c-myb cells also showed a myeloid phenotype. Additionally, c-myb cultures were more heterogeneous and contained mainly granulocytes that resembled promyelocytes.

The c-myb- and v-myb-cells blastoderm and bone marrow were then characterised by antibodies specific for myeloid or erythroid surface antigens and flow cytometry, as well as for lineage specific markers including GATA-1 (erythroid), C/EBPβ (NF-M; myeloid/monocytic) and Mim-1 (granulocytic/promyelocytic). As expected from the cytochemical staining, blastoderm derived bFGF/c-myb- and bFGF/v-myb-cells expressed the erythroid specific surface antigen JS4 and no detectable levels of the myeloid antigens MC51-2 and MC47-3. In addition, cells expressed high levels of transferrin receptor detected by JS8 monoclonal antibody which is particularly high in erythroid progenitors. In contrast, bone marrow derived c-myb- and v-myb-cells expressed the myeloid antigens MC51-2 and MC47-3 and no JS4-antigen. These cells also expressed moderate levels of transferrin receptor which is routinely observed in highly dividing cells and is augmented by transferrin present in culture medium.

To further extend these studies, cells were analysed by Western blotting for expression of lineage specific markers. Blastoderm derived bFGF/c-myb- and BFGF/v-myb-cells expressed high levels of the erythroid marker GATA-1 while the myeloid marker C/EBPβ was absent. In bone marrow derived cells C/EBPβ was highly expressed and GATA-1 was undetectable. Interestingly, bone marrow derived c-myb-cells cells contained high levels of the promyclocytic Mim-1 protein. Mim-1 protein expression steadily increased and at later stages of culture reached levels that were readily detectable by gel electrophoresis and Coomassie blue staining.

In conclusion, bFGF together with c-Myb or v-Myb induced self-renewal of primitive erythroid progenitors from early chick embryos while with bone marrow cells an outgrowth of myeloid cells was observed.

bFGF and v-Myb Cooperate to Induce Self-Renewal and Extended Lifespan of Erythroid Progenitors

The proliferative potential and lifespan of bFGF/v-myb-cells were analysed in long-term suspension cultures and in colony assays. Cells in suspension cultures reached 50 (and even more) population doublings corresponding to 108 cells (FIG. 2A). This lifespan dramatically exceeds that of normal chicken erythroid progenitors (Dolznig et al., 1995, Cell Growth Differ, 6 1341-1352). Another striking feature of these cells is their high doubling rate of 17 h per division as compared to 22-24 h for normal erythroid progenitors (Dolznig et al., 1995, Cell Growth Differ, 6 1341-1352). Furthermore, cells displayed a high clonogenic potential (Data not shown).

In the course of different experiments, bFGF/v-myb-cells at early time points of culture exhibited also the myeloid surface antigen detected by MC51-2 antibody (FIG. 2C, day 12). The proportion of MC51-2 positive cells gradually decreased with time when the culture became more homogenous. By day 18 of culture no MC51-2 specific signal was observed and only erythroid specific markers were present (FIG. 2C).

Finally, the effects of different growth factors (bFGF, SCF and TGFα) on the phenotype of blastoderm derived bFGF/v-myb-cells was studied. As summarised in FIG. 2B proliferating erythroid bFGF/v-myb-cells were obtained only in the presence of bFGF and combinations thereof. SCF or TGFα alone, or no factor yielded an outgrowth exclusively of myeloid cells that expressed MC51-2 surface marker. Upon bFGF withdrawal bFGF/v-myb-cells cells stopped proliferating and eventually died, demonstrating that they are strictly dependent on bFGF.

bFGF Efficiently Blocks Erythroid Differentiation and Promotes Proliferation bFGF/v-myb-Cells

Next, we assessed the ability of bFGF/c-myb- and bFGF/v-myb-cells to undergo terminal differentiation into erythrocytes. Cells were induced to differentiate by anemic serum (as a source of erythropoietin) and insulin, and evaluated by cell morphology, cell size and haemoglobin content. Surprisingly, v-Myb did not block terminal differentiation despite elevated v-Myb protein levels in these cells (FIG. 3) and these cells differentiated into fully mature erythrocytes after 3-4 days. Without factors cells showed a propensity to differentiate but stopped dividing and eventually died, while control cells grown with bFGF remained immature and retained their potent growth characteristics.

Since growth and survival of bFGF/v-myb-cells is strictly dependent on bFGF. we investigated the influence of bFGF on terminal differentiation. Remarkably, bFGF/v-myb-cells, induced to differentiate in the presence of bFGF were completely blocked in their ability to undergo differentiation and kept their self-renewing property and immature phenotype. This result was further supported by measuring other differentiation parameters including cumulative cell numbers, cell volume and haemoglobin content (FIG. 3). Following differentiation induction in the absence of bFGF there was an initial phase characterised by a higher rate of proliferation (FIG. 5B) and then cells stopped dividing after 3 to 4 days. Concomitantly cells reduce their volume and accumulate haemoglobin (FIG. 3). In the presence of bFGF however, bFGF/v-myb-cells continued to proliferate at a high rate (FIG. 3), retained the volume of immature cells of approximately 500 μl and did not accumulate haemoglobin.

bFGF Mediated Mitogenic Signal in bFGF/v-myb-Cells

To gain further insights into the signalling pathways emanating from the FGFRs expressed in bFGF/v-myb-cells, two representatives members of the FGF family, aFGF (acidic FGF, FGF-1) and bFGF were compared in cell proliferation assays. As shown in FIG. 4 nanogram concentrations of bFGP induced a maximal stimulation, whereas 100 times higher levels of aFGF stimulated bFGF/v-myb-cell proliferation only marginally if at all.

The lack of a mitogenic response to aFGF led us to explore the effect of heparin that is known to enhance high affinity binding of FGF-ligands to cognate receptors with high affinity and to augment the mitogenic potential of aFGF. There was no further increase in the bFGF proliferative response by heparin while a clear enhancement of the mitogenic signal was induced by aFGF. However, even 10 limes higher concentrations of bFGF plus heparin were still an order of magnitude less effective in stimulating bFGF/v-myb-cell proliferation than bFGF alone (FIG. 5A). This suggests that bFGF most likely represents the natural ligand or at least a prototype of FGF factors that are active on bFGF/v-myb-cells.

PLCγ-Signalling Pathway is Involved in the Mitogenic Signal Induced by bFGF

The phosphorylation status of downstream signalling substrates of ligand activated FGFRs provided only limited information on the pathways involved in the mitogenic response to bFGF. Therefore, specific inhibitors of signalling molecules die affecting various pathways were employed. FIG. 5B shows that neither Ras inhibitor FTS (inhibitor of famesylation) nor src family inhibitor PP-2 had any effect on bFGF induced bFGF/v-myb cell proliferation even at concentrations 10 times higher than routinely effective. The phosphatidylinositol 3-kinase (PI3-kinase) inhibitor Worthmannin was also ineffective while the MEK inhibitor PD 98059 was partially inhibitory only at higher concentrations (FIG. 5B). Importantly, the PLCγ-inhibitor U-73122 dramatically reduced the bFGF induced proliferative response even at low concentrations (FIGS. 5B, C) indicating that PLCγ is involved in transmitting the mitogenic signal induced by bFGF.

It has been shown that PLCγ-signalling leads to activation of protein kinase C (PKC) via diacylglycerol (DAG) and increases intracellular calcium by inositol triphosphate (Ins3P). Increased calcium has pleiotropic effects and can result in activation of calmodulin dependent protein kinase (CaMKII) and calcineurin (phosphatase PP2B). Therefore inhibitors of calcium dependent pathways were also tested. The PKC inhibitor GF109203X reduced bFGF induced bFGF/v-myb cell proliferation by 30%, while the reduction by the calcineurin inhibitors cyclosporine A and cypermethrin was 45% (FIG. 5B). Sixty per cent of inhibition was measured in the presence of KN-62, a specific inhibitor of CaMKII. Taken together these data suggest that in bFGF/v-myb-progenitors PLCγ might be one of the important molecules that triggers mitogenic signalling emanating from bFGF while further downstream signalling events involve multiple transduction pathways.

### LEGENDS TO THE FIGURES

FIG. 1

bFGF- and Myb-proteins support the proliferation of primitive erythroid progenitor cells. (A, B) Growth of the v-myb- and c-myb-cells from blastoderm and bone marrow in the presence of bFGF and SCF was monitored by daily counting and plotted as cumulative cell numbers. Control, non-infected cells.

FIG. 2

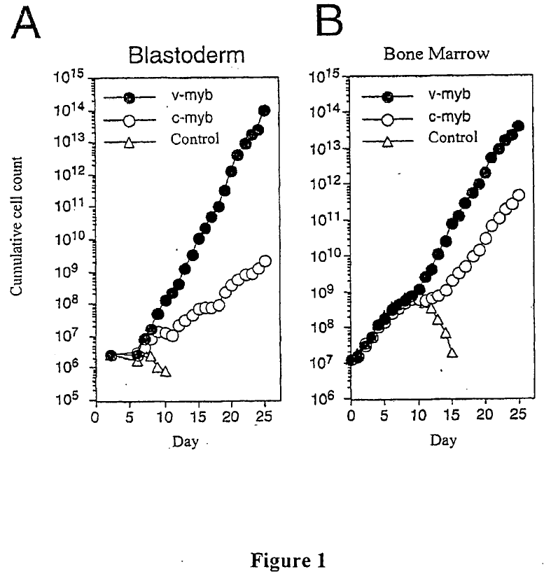

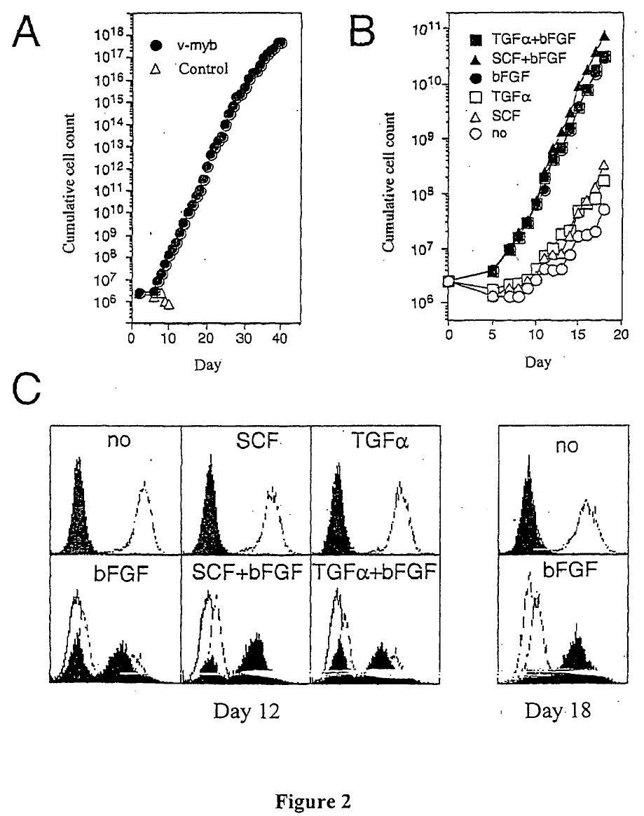

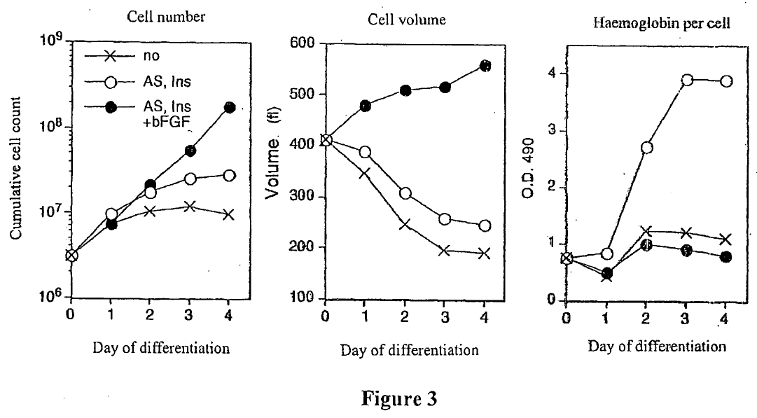

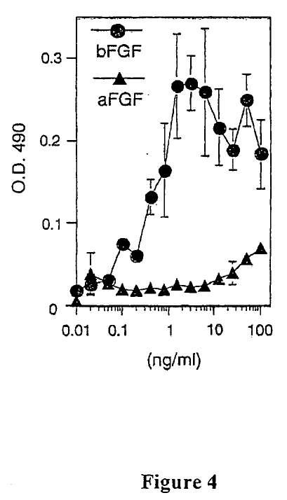

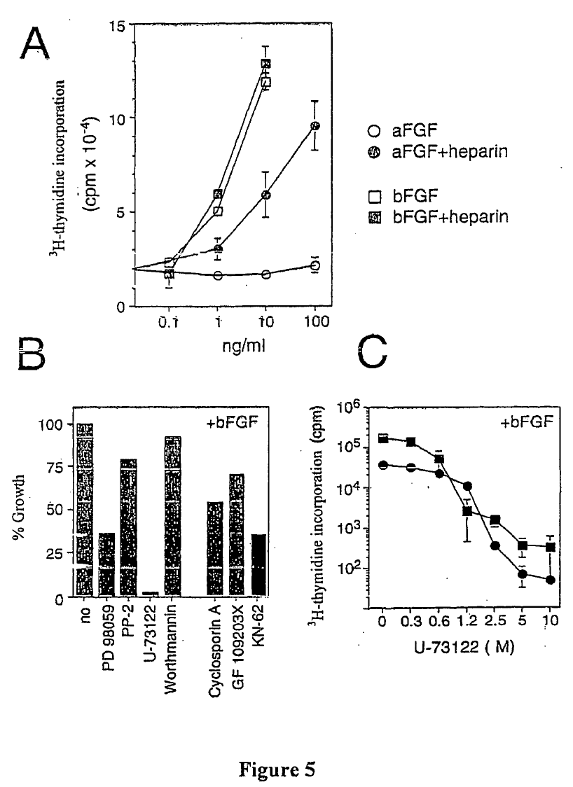

Characterisation of bFGF/v-myb-cells for extended lifespan and cell-surface antigen expression

(A) Growth curve of bFGF/v-myb cells (closed circles) cultivated in the presence of of bFGF for more than 50 generations. Control, non-infected cells (open triangles).

(B) Growth factor requirements of bFGF/v-myb-cells. The cumulative number of cells gown with various factors and combinations thereof are shown. Closed and open symbols refer to cells with an erythroid and myeloid phenotype, respectively.

(C) FACS-analysis of cells at day 12 and 18 of culture (same cells as in B,C) using myeloid (grey) and erythroid (black) specific monoclonal antibodies (MC51-2 and JS4, respectively)

FIG. 3

Differentiation properties of bFGF/v-myb erythroid progenitor cells. Kinetics of the different differentiation parameters of bFGF/v-myb-cells. The number of cells, the cell volume, and the amount of haemoglobin were estimated during the 4-day differentiation in absence (AS, Ins) or presence of bFGF (AS, Ins+bFGF) or without factor (no). The cells remaining in bFGF are only shown as a control (bFGF).

FIG. 4

FGF—signalling in erythroid FGF/v-myb-progenitor cells.

FGF—dose response measured by cell proliferation assay (MTT). Serial dilutions of aFGF and bFGF, respectively, starting at 100 ng/ml are shown.

FIG. 5

Response of bFGF/v-myb-cells to aFGF and bFGF, and effects of specific inhibitors on FGFR-signalling.

(A) Dose response curve of aFGF and bFGF in the presence or absence of heparin is shown. Cell proliferation was measured by ³H-thymidine incorporation assay.

(B) The bFGF/v-myb-cells were stimulated with bFGF and with specific inhibitors (for details see methods). Cell proliferation was measured in (A) per cent of proliferation in response to bFGF (25 ng/ml) and specific inhibitor is shown. Proliferation without inhibitor was arbitrarily set 100%.

(C) Dose response curve of the PLCy inhibitor U-73122 on bFGF/v-myb-cells in the presence of bFGF (25 ng/ml). Cell proliferation was measured by ³H-thymidine incorporation assay as in (A). Two independent experiments arc shown.

1. Transformed blood cells for the elucidation of factors, components, that influence the formation and differentiation of blood cells, characterised in that they contain Myb, in particular v-Myb, and growth factors interacting with Myb.

2. Transformed blood cells according to claim 1, characterised in that they contain Myb as part of the AMV (Avian Myeligblastosis Virus) or as part of a recombinant retrovirus that contains different alleles of Myb.

3. Transformed blood cells according to claim 1 and 2, characterised in that they contain the growth factor bFGF.

4. Transformed blood cells according to claim 1 and 2, characterised in that they contain the growth factor SCF.

5. Transformed blood cells according to claim 1 and 2, characterised in that they contain the growth factor EGF.

6. Transformed blood cells according to claim 1 and 2, characterised in that they contain the growth factor TGF alpha.

7. Transformed blood cells according to claim 1 and 2, characterised in that they contain another ligand that interacts with myb instead of the growth factor.

8. Method for the production and cultivation of the cells, characterised in that
a) hematopoietic precursor cells are isolated from, e.g., bone marrow, early embryos or stem cells,
b) myb cDNA is transferred, and
c) the growth and the proliferation of the myb-cells obtained occurs under specific culture conditions, preferably in the presence of growth factors.

9. Use of the cells according to claim 1 to 7, characterised in that they are used for high throughput screening assays (HTS) for the identification of small molecules or proteins that can influence the fate, the differentiation, the proliferation, and self-renewal, the growth inhibition, and cell death of the different cells.

10. Use of the cells according to claim 1 to 7, characterised in that they are used for the identification of specific agonists and/or antagonists of the FGF signals.
